(19) United States
(12) Patent Application Publication
(10) Pub. No.: US 2024/0219388 A1
(43) Pub. Date: Jul. 4, 2024

(54) Method For Fractionation Of Biomolecules

(71) Applicants: Pierce Biotechnology, Inc., Rockford, IL (US); Life Technologies Corporation, Carlsbad, CA (US); Bender MedSystems GmbH, Vienna (AT)

(72) Inventors: Paul Haney, Beloit, WI (US); Surbhi Desai, Rockford, IL (US); Marie Murakami, Encinitas, CA (US); Penny Jensen, Marengo, IL (US); Brian Steer, San Diego, CA (US); Kelli Feather-Henigan, Winnebago, IL (US); Syrus Jaffe, Carlsbad, CA (US); Brian Webb, Roscoe, IL (US); Korin Pathammavong, South Beloit, IL (US); Ricarda Heintz, Wien (AT); Jordan Thompson, Murrieta, CA (US); Jeremy Weaver, Rockford, IL (US); Carina Wimer, San Diego, CA (US); Andrew Westergren, Murrieta, CA (US); Kai Chun Chen, Encinitas, CA (US)

(21) Appl. No.: 18/391,779

(22) Filed: Dec. 21, 2023

Related U.S. Application Data
(60) Provisional application No. 63/477,941, filed on Dec. 30, 2022.

(51) Int. Cl. G01N 33/573; C12N 15/10; G01N 1/34; G01N 33/68

(52) U.S. Cl. CPC G01N 33/573; C12N 15/101; G01N 1/34; G01N 33/68; G01N 2333/90203; G01N 2333/98

(57) ABSTRACT

Methods for fractionating a sample of biomolecules of the disclosure comprise (a) introducing a sample into a separating column; (b) separating biomolecules in the sample by molecular weight into a plurality of fractions along the separating column; and (c) placing the separating column into successive engagement with a plurality of wells and advancing into each of the wells one or more of the corresponding plurality of fractions. Methods of the disclosure further comprise detecting and identifying biomolecules in the fractions using microparticles to bind to the fractions, and novel approaches to pool the fractions followed by assays—such as immunoassays—that allow high throughput and automation capabilities.

### CROSS-REFERENCE TO RELATED APPLICATIONS

The present application claims priority to and the benefit of U.S. patent application No. 63/477,941, “Method For Fractionation Of Biomolecules” (filed Dec. 30, 2022). All foregoing applications are incorporated herein by reference in their entireties for any and all purposes.

### TECHNICAL FIELD

The present invention relates to methods and tools for analysis of biomolecules in a sample. The invention provides novel methods for separation and/or fractionation of small samples of biomolecules using gel electrophoresis/electroelution for detection using immunoassays of biomolecules without requiring the step of transferring of biomolecules to membranes.

### BACKGROUND

Western blotting is a widely used technique for monitoring protein expression and regulation. Although useful, the technique also has certain limitations, which include limited throughput, difficulty with implementing the technique in a multiplexed manner, limited ability to reuse the technique, and also the need for time-consuming, hands-on process steps.

In Western blotting, proteins are electrophoretically separated in a gel (typically located in a cassette), after which the separated proteins are retained in the gel; as is known to those in the field, separation gels can be fragile and difficult to handle. The gel is then removed from the cassette and placed on a membrane and filter stack, and the gel is then moved to a further device to electrophoretically transfer of the proteins in the gel onto the membrane. An exemplary such approach is described in U.S. Pat. No. 10,274,485 and European Patent No. 2 737 319 (incorporated herein by reference in their entireties for all purposes), which describe electrophoretically separating proteins in a gel, transferring the proteins to a membrane, mechanically cutting columns out of a protein-bearing membrane, and then further cutting those columns into a number of narrow strips that are then moved again to a different location for further processing, as shown in FIG. 1. Such a process, however, requires multiple instruments, repeated handling and movement of fragile separation gels and membrane, and mechanical cutting of membranes into narrow strips. Further, existing approaches' reliance on moving and handling fragile slab gels and membranes in turn makes these processes being ill-suited for automated operation and also makes the existing processes difficult to implement in a multiplexed way. Northern and Southern blot techniques suffer from the same challenges as Western blots; although Northern and Southern blots address nucleic acids as targets instead of proteins as targets, Northern and Southern blots nonetheless involve tedious, labor-intensive handling of gels and membranes.

Accordingly, there is a long-felt need in the art for improved Western blot-based techniques for monitoring protein expression and regulation, in particular for techniques that involve less movement of gels, membranes, and reagents; that require less user labor; and that are more compatible with automation.

### SUMMARY

The disclosed technology provides an improved alternative to traditional Western, Northern and Southern blotting approaches in which biomolecules in a sample—such as cell/tissue lysate—are separated by size in a gel, transferred to a membrane, and then probed—for example, immunoprobed with an antibody-based detection method—to measure the amount of a specific biomolecule in the sample relative to other samples. As explained herein, the disclosed technology provides a significant improvement over the existing technologies, and does so in a way that makes detection of multiple biomolecules of interest significantly less labor-intensive and also more directly compatible with automation. Biomolecules that can be analyzed by the disclosed technology include proteins, nucleic acids (including DNA, RNA, etc.), carbohydrates, and lipids.

In meeting the described long-felt needs, the disclosed technology provides a method for fractionating a sample of biomolecules, comprising (a) introducing the sample into a separating column; (b) separating biomolecules in the sample by molecular weight into at least n fractions along the separating column; and (c) placing the separating column into successive engagement with first through nth wells and advancing into each of the first through nth wells the corresponding first through nth fraction.

Embodiments of the disclosed technology further comprise detecting and identifying biomolecules that are fractionated from a sample (such as a cell or tissue lysate). In some embodiments detecting and identifying biomolecules comprises contacting one or more of the n fractions with a distinct microparticle each to form one or more distinct fractions, wherein each distinct fraction is different from other distinct fractions. Embodiments can further comprise combining the x distinct fractions to form a fraction pool. Pre-labeled or labeled fractions may be contacted with one or more buffers. Embodiments can further comprise determining the presence or absence of a biomolecule in portions/aliquots of the fraction pool, by binding the biomolecules in the pooled fractions with one or more known labeled molecules that can specifically bind to the biomolecules (such as primary and/or secondary antibodies, complementary nucleic acid probes, or any molecule with specific affinity for the biomolecule).

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1 depicts an existing Western blot-based fractionation and assay process.

FIGS. 2A and 2B depict an exemplary workflow according to the disclosed technology for fractionating and assaying biomolecules from a sample.

FIG. 3A depicts a schematic (left panel) of a single separating column as contemplated by the disclosed technology, and an image (right panel) of a separating column with a partially separated molecular weight ladder that has not yet been eluted from the column and FIG. 3B provides example separation columns and a conductive collection plate.

FIGS. 4A and 4B depict Western blot analysis and validating protein fractions after sample fractionation using the separating column and fractionation methods of the present disclosure. FIG. 4A provides a validation of the disclosed technology and depicts an image of total protein detection on a membrane of protein fractions prepared from a protein sample fractionated using the separating column and methods of the disclosed technology; SDS-PAGE was performed followed by transfer to a membrane, and a classic Western blot was used to analyze the separation pattern of the well-collected material. FIG. 4B provides a validation of the disclosed technology and depicts a Western blot showing detection of p23, GAPDH, and HDAC1, on a membrane containing fractions of a protein sample fractionated using the separating column and methods of the disclosed technology.

FIG. 5 illustrates the effect on assay signals from varying the surfactant in an example dilution buffer when using the disclosed technology.

FIG. 6 illustrates the effect on assay signals from varying the composition of an example dilution buffer when using the disclosed technology.

FIG. 7 illustrates the effect on assay signals from varying the detergent in an example dilution buffer when using the disclosed technology.

FIG. 8 illustrates the application of the disclosed technology to an application in which nucleic acid—such as DNA or RNA—is the biomolecule target of interest.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

The following embodiments are illustrative only and do not limit the scope of the disclosed technology. Any part or parts of any embodiment can be combined with any part or parts of any other embodiment or embodiments.

### Discussion

The disclosed technology allows for analysis of multiple biomolecules within one biological sample, such as a cell lysate, without the need for repetitious and labor intensive Western/Southern/Northern blotting workflows. Although several of the examples below are described with respect to Western Blotting and protein biomolecules, these illustrative examples and embodiments do not limit the scope of the disclosed technology and other biomolecules such as nucleic acids, carbohydrates, and lipids can be detected and analyzed by the disclosed technology.

In one embodiment, wherein the biomolecule is a protein, the disclosed technology allows for multiple proteins within one biological sample, such as a cell lysate, to be analyzed without the need for repetitious and labor intensive Western blotting workflows or for complex “one pot” multiplexed Luminex™ immunoassays.

As described herein, in existing Western blot approaches proteins are electrophoretically separated in a gel—typically located in a cassette—after which the separated proteins are retained in the gel; as is known to those in the field, separation gels can be fragile and difficult to handle. The gel is then removed from the cassette and placed on a membrane and filter stack, and the gel is then moved to a further device to electrophoretically transfer of the proteins in the gel onto the membrane, for example, at a 90-degree orientation to the path of protein separation.

One such approach is presented by U.S. Pat. No. 10,274,485 and European Patent No. 2 737 319 (incorporated herein by reference in their entireties for all purposes), which describe electrophoretically separating proteins in a gel, transferring the proteins to a membrane, mechanically cutting columns out of a protein-bearing membrane, and then further cutting those columns into many narrow strips.

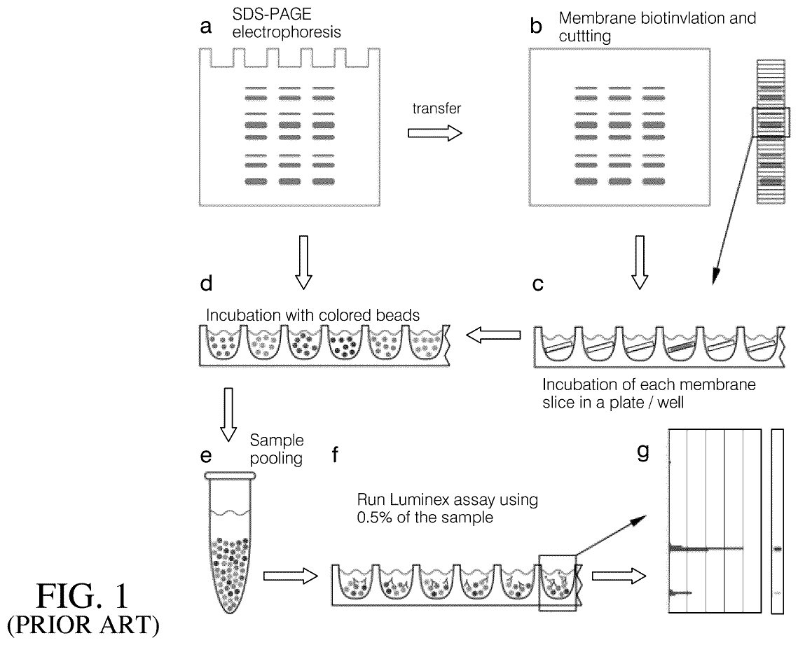

Such a labor-intensive approach is shown by FIG. 1. As shown, in existing approaches, proteins are electrophoretically separated in a gel, as shown in step a of FIG. 1. The gel is then be moved to membrane and filter stack and transferred to a different device for electrophoretic transfer of the proteins in the gel onto the membrane, for example, in a 90-degree orientation to the path of protein, as shown in step b of FIG. 1. This requirement for movement and handling of slab gels and membranes makes the process fragile and not conducive to being executed in an automated fashion. Further, as shown by step b and step c of FIG. 1, the existing process involves mechanically cutting columns out of the protein-bearing membrane, and then further cutting those columns into many narrow strips, which strips are then further transferred to plates/wells for further processing, for example, via incubation with colored beads, sample pooling, and performance of a LUMINEX™ assay to ascertain the presence of proteins of certain weights in the original sample as shown in step c, step d, and step e of FIG. 1.

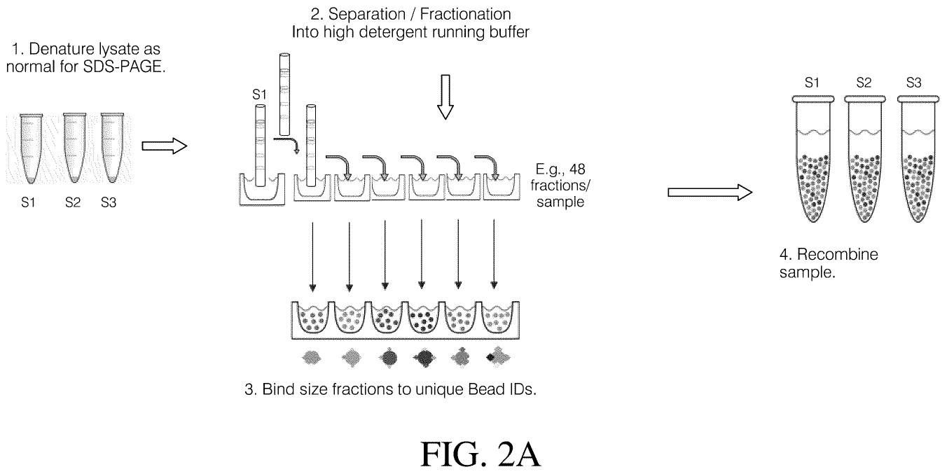

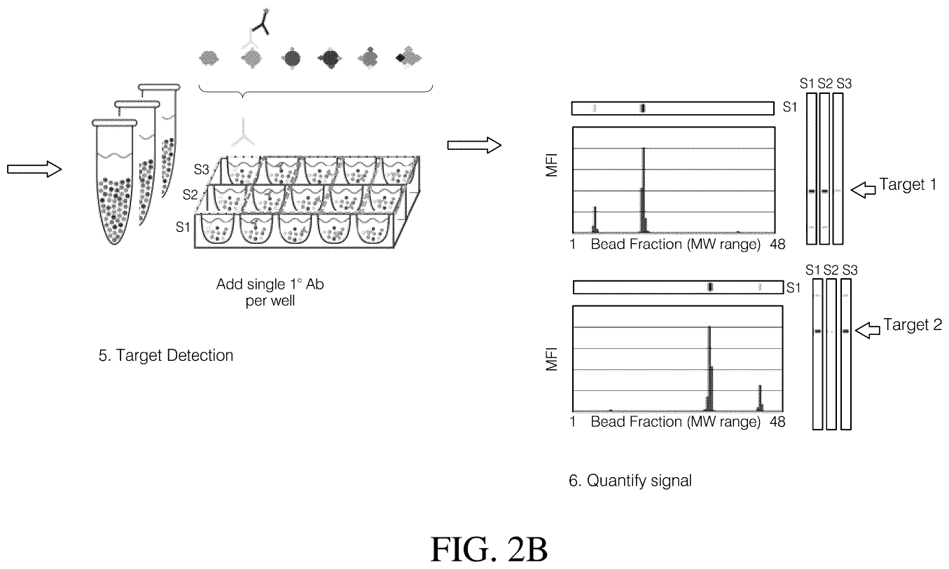

By comparison, in the disclosed technology, proteins of a complex biological sample are separated in size by gel electrophoresis, for example, SDS-PAGE electrophoresis, as depicted in exemplary FIGS. 2A and 2B. It should be understood that FIGS. 2A and 2B are illustrative only and are non-limiting. In particular, it should be understood that a wide variety of different SDS-PAGE separation chemistries are available and can be used with the disclosed technology. Other separation methods—such as native gel electrophoresis—are also suitable.

As shown, proteins can be denatured—as shown in step 1 in FIG. 2A—and the proteins can be introduced into a separation column or columns, as shown in FIG. 2A, step 2, which columns include therein a separation matrix. The electrophoresis can be conducted such that the proteins migrate completely through the separation matrix—the separation matrix can be present in a separation column—and exit the separation matrix on the anode side, where the proteins are collected in multiple separate fractions—for example, 48 fractions—over a period of time, as shown in step 2 of FIG. 2A.

In an example application of the disclosed technology, one can separate proteins of a molecular weight of, for example, 10˜ 200 kDa into 48 fractions. This number can be used because it allows sufficient resolution and prevents spreading of proteins across too many fractions. Different ranges of molecular weight can, however, be collected by modifying the dwell time in each fractions. Similarly, more than 48 fractions or fewer than 48 fractions can also be collected, for example, 96 fractions can be collected, as one example. A smaller number of fractions can be collected if a user may prefer to focus on a smaller molecular weight window or if lower resolution were needed for a given investigation.

In some embodiments, a separation column can be a single-use component. A separating column can include glass, plastic, or even both materials. Reusable columns, such as reusable capillary electrophoresis columns, can also be used with the disclosed technology. In some embodiments, a separating column can comprise one or more of the following: a region for sample addition, a region for sample/biomolecule separation, an area to hold electrophoresis buffer, and an electrical contact; the column can also or as an alterative include a means for electrical contact. In some embodiments, a separating column can be filled with a solid or liquid based separation matrix, such as but not limited to polymerized acrylamide (SDS-PAGE), linear acrylamide, PDMA, or dextran. Separating columns can be cylindrical, square, or rectangle in shape. In some embodiments, exemplary internal diameter dimensions of the region where biomolecule separation occurs can be in the range of from about 0.1 mm-1 mm.

The collection can be done in different ways; one such way is by placing an end of a small electrophoresis column into engagement with a sample well—for example, in a multi-well plate, such as a 384 well plate—and supplying a current. The current can be supplied, for example, by having an anode wire deposited within the elution buffer or by having the microwell containing the elution buffer be conductive and directly serving as the anode. The separation column can then be moved to a new well; this can be effected every few seconds to minutes, depending on the user's needs and the characteristics of a given sample. As will be understood, a cathode electrode can be present so as to complete the circuit to effect the separation. Further, the collection well—as described elsewhere herein—can include an electrode and/or itself be conductive. The separating column can comprise at least a portion that is conductive.

In this way, each well's fraction will contain a different mixture of proteins of a shared molecular weight range that exit the gel at the same time, with the lowest molecular weight proteins being in the earliest-eluted fractions, and the largest molecular weight proteins being presented in later-eluted fractions. The gel matrix formulation, the electrophoresis buffers, the applied electrical potentials—for example, voltage, current—the fraction collection times, and the number of fractions can influence how many proteins and the molecular weight range found in each fraction.

It should be understood that engagement between a separation column and a well can be performed in an automated fashion, for example, by moving the separation column from a first well to a second well when an automated system has detected the movement of a particular molecular weight standard or other marker within or out of the separation column, thereby signifying the passage of proteins having a molecular weight in accordance with that particular molecular weight standard. In this way, successive fractions of increasing-weight molecules can be eluted according to size into successive wells, and the process can be controlled in an automated fashion.

After collection, each fraction is then bound to a specific pool of beads or other detection species; the detection species for each fraction are suitably distinct and can be identified from one other based on, for example, color, size, pattern, labels/barcodes, or other factors, as shown in step 3 of FIG. 2A. Although LUMINEX™ beads were used in the provided examples, it should be understood that LUMINEX™ beads are not the exclusive method for tracking protein fractions. The buffer in which the proteins are bound to the beads can influence the process, as the buffer can influence bead binding efficiency, the form of the protein on the bead, and detection of the protein by antibodies in a later step.

After proteins are bound to the beads, the distinct bead fractions can be recombined as shown in step 4 of FIG. 2A, which recombination can be performed with or without initial washing. This recombination in turn recreates the full sample, but the molecular weight of each protein is now encoded to a small range of bead IDs/profiles. These combined bead pools can include enough beads and sample to allow for the approach to support multiple separate immunoassays.

To conduct further immunoassays, a small fraction of the final bead pool—which can be, for example, 1%—is placed in independent assay reaction tubes/wells and antibody against a protein of interest is added with a different primary antibody in each assay reaction, as shown in step 5 of FIG. 2B. The signals from each independent assay can then be collected and quantified, shown in step 6 of FIG. 2B.

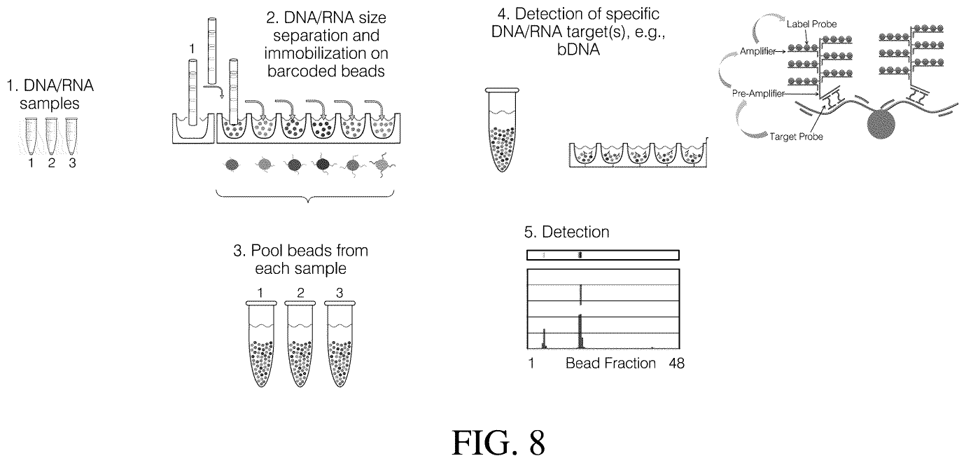

Another depiction of the disclosed technology is provided in FIG. 8. As shown, a nucleic acid biomolecule sample at step 1 is electrophoretically separated in a separation column, as shown in step 2. As depicted, fractions of nucleic acid in the sample are advanced into a series of collection wells, with each well containing a different fraction; fractions can be defined by nucleic acid size/weight. As shown in step 2, each distinct fraction of nucleic acid can be contacted with a distinct microparticle—such as a microbead bearing a fluorescent label or the like—to form labeled fractions. The labeled fractions can then be pooled, for example, as in step 3). Detection of specific nucleic acid targets can then be performed—shown at step 4—by, for example, using a branched DNA (bDNA) or other detection approach. Further analysis—shown at step 5—can then be performed, for example, via a LUMINEX™ approach to assess the presence or absence of certain nucleic acids in the original sample.

Accordingly, methods for fractionating a sample of biomolecules by the disclosed technology provide methods for efficiently separating biomolecules comprising (a) introducing a sample into a separating column; (b) separating biomolecules in the sample by molecular weight into a plurality of fractions along the separating column; and (c) placing the separating column into successive engagement with a plurality of wells and advancing into each of the wells one or more of the corresponding plurality of fractions. Methods of the disclosure further comprise detecting and identifying biomolecules in the separated fractions using microparticles to bind to the fractions, and novel approaches to pool the microparticle associated fractions, further followed by immunoassays and/or other biomolecule specific assays that allow high throughput, multiplexing, and automation capabilities.

The disclosed technology thus has a number of key applications. As but one example, the disclosed technology can be used by researchers to more easily study protein regulation pathways in an automated fashion that significantly improves upon the amount of data typically generated for one sample by classical protein immunodetection methods such as western blotting.

### Advantages

As explained elsewhere, existing approaches have many limitations in terms of throughput, multiplexing limitations, reuse, and hands-on time requirements. This is due to those approaches' complex multistep workflow, which requires multiple instruments and the repeated handling and movement of fragile separation gels and membranes, as shown in FIG. 1.

The disclosed technology provides a number of changes from standard membrane-based Western blotting, Northern blotting or Southern blotting.

First, the disclosed technology allows each sample to be assayed for tens to hundreds of target proteins without limitations from secondary antibody cross-reactivity or from off-target interference from other targets. In classical Western blotting, one is limited to how many proteins one can detect in a lane by the use of primary and secondary antibodies. In the classical existing methods, because the full lane is being immunoprobed at one time, one cannot reliably probe for multiple targets. Similar advantages are provided by the technology for analysis other type of biomolecules such as DNA, RNA, and others.

In the disclosed method, the sample is not confined to a lane on a membrane but instead is bound to a pool of beads which has enough material for multiple assays. The disclosed approach thus turns one sample lane into tens to hundreds of “sample lanes” that are then all probed with specific antibodies—or even with complementary nucleic acid probes—each as an individual assay in a well. This approach dramatically increases the number of targets that can be assayed per sample, increases throughput, and eliminates the risk of cross-reactions and interference common to multiplex assays with multiple antibody specificities in one assay.

Second, the disclosed technology is more compatible with automation as compared to classic Western/Northern/Southern blotting, as gel and membrane handling is not required. In existing methods—as shown in FIG. 1—proteins are electrophoretically separated in a slab gel in a cassette where the proteins are retained in the gel. The gel then has to be removed from the cassette and placed on a membrane and filter stack and moved to a different device to electrophoretically transfer of the proteins in the gel onto the membrane in a 90-degree orientation to the path of protein separation. This reliance on the movement and handling of slab gels and membranes makes existing processes fragile and ill-suited to performance in an automated way.

By contrast, in the disclosed technology, the biomolecules—which can be, for example, proteins or nucleic acids—are not left in the gel after separation and then transferred to a membrane. Continued electrophoresis and movement of the separation consumable—which can be, for example, a separation column—along a microwell plate allows the biomolecules to be directly eluted out of the separation matrix; such a matrix cab be, for example, SDS-PAGE for proteins. The foregoing can be effected without manual involvement or the need to capture biomolecules onto a membrane. In the disclosed technology, the eluted biomolecules can be stabilized in an appropriate buffer and then bound to ID-coded microbeads directly in solution. In this approach, these beads take the place of a membrane in the assay. This in turn greatly improves handling, as all processing is done in a “liquid pool” and is directly compatible with standard pipet offerings, magnetic bead processing and liquid handling automation.

Third, in Western blotting, each sample lane is typically used in a single low-plex experiment. After a sample is on a membrane and immunodetected, the sample is then disposed of. If additional analysis of that sample is desired after the initial results are reviewed, the full experimental workflow—which can require days—must be repeated. In the disclosed technology, however, the bead pool containing the separated and bound sample can be used in aliquots and the bead pool can be stored and a fraction of it used at a later date—for example, 1 month later—to allow additional assays to be performed on a sample without the need to rerun the sample.

### Embodiments

Embodiments of the disclosed technology provide methods for fractionating a sample of biomolecules. The biomolecules may include one or more of a protein, a nucleic acid, a carbohydrate, or a lipid. The sample of biomolecules may include a sample obtained from a cell sample or cell lysate, a tissue sample or tissue lysate, or one or more combinations thereof. The tissue sample may include a mammalian tissue sample. The sample of biomolecules may be prepared using any suitable technique as understood in the art including lysing, linearizing, denaturing, and the like.

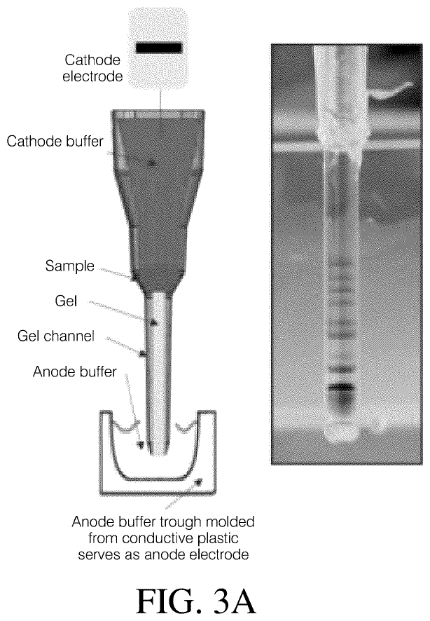

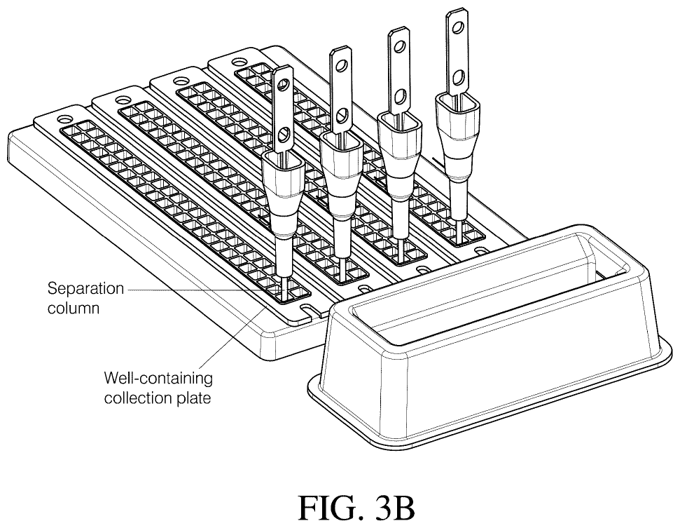

Embodiments of the methods include introducing the sample into a separating column. Exemplary separating columns can include a channel that is at least partially filled with a solid or liquid separation matrix, for example, polymerized acrylamide (SDS-PAGE), linear acrylamide, PDMA, or dextran, as but some non-limiting examples. A separation column can be circular—such as a cylinder in shape, square, oval, or rectangular in conformation; a separating column can have an internal diameter in the range of from about 0.1 mm to about 1 mm. A separating column can include a region for sample addition; a column can also have an area to hold electrophoresis buffer. A column can also include (or accommodate) an electrical contact—for example, at the top or upper portion of the column—for the electrophoresis buffer. A separating column can have a constant inner diameter, but this is not a requirement, as a separating column can have an inner diameter and/or inner cross-sectional shape that varies along the length of the separating column. As but one example, the inner diameter can taper in either direction along the length of the separating column. As shown in illustrative, non-limiting FIG. 3A, a separating column can be a column with an open top and an open bottom and can include an upper portion and a lower portion. The upper portion can have a wider diameter than the lower portion for receiving samples, buffers and the like. The upper portion can include a maximum diameter at the top of the separating column for loading a sample including a biomolecule sample and one or more buffers including cathode buffers into the column. In some embodiments, the upper portion has a funneled configuration for receiving said samples. The lower portion can contain a separating medium and can have a constant outer diameter with an open bottom for dispensing separated samples. In some embodiments, the bottom of the lower portion has a beveled configuration. The separating column can be made of one or more suitable materials including, for example glass, plastic, or a combination thereof.

In some embodiments, the upper portion or lower portion are made of the same material. In some embodiments, the upper portion and lower portion of the column are made of different materials. For example, in some embodiments, the upper portion is made of one or more plastics including polyethylene, polypropylene, and the like, and the lower portion is made of glass.

In some embodiments, the top of the upper portion of the separating column is configured for engaging with one or more electrode leads. In some embodiments, the upper portion of the separating column is configured for engaging with one or more leads of power supply. In some embodiments, the upper portion of the separating column is configured for engaging with one or more manifolds including automation manifolds for loading samples, buffers and the like and applying a charge to the top portion of the column. A portion—such as the upper portion—of the column can be conductive, for example, comprise a conductive plastic. Similarly, the collection well can itself be conductive, for example, comprise a conductive plastic.

In some embodiments, the separating column includes a separating medium. The separating medium can include, for example, a stacking gel, a separating gel, a liquid matrix, and/or one or more combinations thereof. In some embodiments, the separating gel includes a Tris-glycine gel, a Bis-Tris gel, a Tris-acetate gel, a Tricine gel, a polyacrylamide gel, an acrylamide gel, an agarose gel, or any combination thereof.

A separating gel can include a single-percentage gel or a gradient gel. The single-percentage separating gel can include a monomer or polymer percentage of from about 5% to about 25%. For example, the percentage of monomer or polymer can include about 5%, about 7.5%, about 8%, about 10%, about 12%, about 15%, about 20%, about 25%, and any and all increments therebetween.

A gradient separating gel can include a percentage range of monomer or polymer of from about 3% to 8%, from about 3% to 12%, from about 4% to 12%, from about 4% to 15%, from about 4% to 16%, from about 4% to 20%, from about 8% to 16%, or from about 10% to 20%.

In some embodiments, the stacking gel includes a polyacrylamide gel. The stacking gel may include a single percentage of polyacrylamide in the range of from about 2% to about 10%. For example, the stacking gel may have a polyacrylamide amount of about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, or any intermediate value therebetween. In some embodiments, the separating medium is positioned in the lower portion of the separating column.

In some embodiments, the biomolecule sample is prepared in one or more sample buffers. The sample buffer can include one or more ingredients suitable for preparing the sample for separation. For example, in some embodiments, the sample buffer includes one or more ingredients for linearizing proteins or nucleic acids. In some embodiments, the sample buffer includes one or more ingredients for de-aggregating or separating any proteins, nucleic acids, lipids or carbohydrates in the biomolecule sample.

The sample may be added to the column in a volume of up to about 0.5 μL, from about 0.5 μL to about 1 μL, from about 1 μL to about 1.5 L, from about 1.5 μL to about 2 μL, from about 2 μL to about 2.5 μL, from about 2.5 μL to about 3 μL, from about 3 μL to about 3.5 μL, from about 3.5 μL to about 4 μL, from about 4 μL to about 4.5 μL, from about 4.5 μL to about 5 μL, and any and all increments therebetween. In some embodiments, after the sample is loaded into the top portion of the separating column, a volume of a cathode buffer is added to the separating column. In some embodiments, the cathode buffer forms a fluid seal on the top of the separating column. The one or more cathode buffers can include a running buffer such as one or more standard running buffers as understood in the art.

In some embodiments, the sample buffer contains one or more molecular weight markers as understood in the art including for example a size standard, a dye, a fluorescent marker, a colorimetric marker, or any combination thereof. The one or more molecular weight markers can include one or more protein standards, protein ladders, or other protein molecular weight markers as understood in the art, including for example, one or more of Magic Mark™, PageRule™, BenchMark™, SuperSignal™, iBright™, Spectra™, and the like, including one or more combinations thereof.

An example column is shown in FIG. 3A. As shown on the left, a column can contain a cathode electrode; a cathode buffer; sample—which can be, for example, denatured protein; a separation gel—which can be a type of separation medium—disposed disposed in a gel column; and an anode buffer. A well can be configured to comprise an anode buffer trough; the well can be formed from a conductive plastic or other conductive material so as to act as an anode electrode. The right-hand image of FIG. 3A provides an image of a column according to the present disclosure, with the column containing a partially separated but not yet eluted molecular weight ladder.

FIG. 3B provides example separation columns and an example conductive collection plate. As shown in FIG. 3B, one or more separation columns can be used to advance material—such as separated sample fractions—into one or more wells of a well-containing collection plate. A separation column can be moved between first and nth wells of the well-containing collection plate, and can be operated so as to advance into each of the first through nth wells a corresponding first through nth fraction of separated sample. This can be accomplished by a single separation column; as shown, one can also use multiple separation columns. A separation column can be moveable—for example, with an armature or other element—relative to a well-containing sample collection plate. The well-containing sample collection plate can also be moveable relative to the separation column. The disclosed technology can thus operate with movement of at least one of a separation column and a well-containing collection plate relative to the other of the separation column and the well-containing collection plate. Separation columns can be arranged to move and/or operate simultaneously, for example in a parallel fashion. As an example, a set of multiple separation columns can be moved on concert with one another, with each separation column being associated with a different well-containing collection plate. A well can contain a buffer—such as an anode buffer—and a well can be formed from or otherwise include a conductive material.

Embodiments of the methods include separating the biomolecules in the biomolecule sample along the separating column. The biomolecule sample is separated along the separating column by placing the separating column in an engaged configuration wherein the bottom of the separating column is in fluid communication with a fractionation buffer and the column is engaged with at least one lead for applying an electrical potential—for example, a voltage, and/or a current—across the separating column. The fractionation buffer can include an anode buffer.

In some embodiments, the top of the separating column is configured for engaging with at least one lead including for example a cathode lead. In some embodiments, the well is configured for engaging with at least one lead including for example an anode lead. In some embodiments, the anode lead engages with the well. In some embodiments, the anode lead engages with the fractionation buffer within the well. A portion—such as the upper portion—of the column can be conductive, for example, for example, comprise a conductive plastic. Similarly, the collection well can itself be conductive, for example, comprise a conductive plastic.

The fractionation buffer can include one or more buffers including a Tris-glycine-based buffer, a Tris-Acetate-based buffer, a Tricine buffer, a morpholinopropane-1-sulfonic acid (MOPS) buffer, a 2-(N-morpholino)cthanesulfonic acid (MES) buffer, or one or more combinations thereof. In some embodiments, the fractionation buffer is contained in a priming trough and the separating column is first placed in the priming trough and then an electrical potential is applied across the separating column in order to initiate separation of the biomolecule sample thereby priming the separating column.

In some embodiments, the separating column is placed in a fraction well containing a volume of fractionation buffer. The fraction well can be a well of a multi-well plate where each well contains a volume of fractionation buffer. A multi-well plate can include a 4-well plate, a 6-well plate, an 8-well plate, a 12-well plate, a 24-well plate, a 48-well plate, a 96-well plate, a 384-well plate, or a 1536-well plate.

The volume of fractionation buffer can include up to about 1 μL, from about 1 μL to 2 μL, from about 2 μL to about 3 μL, from about 3 μL to about 4 μL, from about 4 μL to about 5 μL, from about 5 μL to about 6 μL, from about 6 μL to about 7 μL, from about 7 μL to about 8 μL, from about 8 μL to about 9 μL, from about 9 μL to about 10 μL and any and all increments therebetween.

In some embodiments, the separating column placed in the first well is primed. In some embodiments, the separating column placed in the first well is not primed.

In some embodiments, the electrical potential applied across the separating column is a constant voltage. The constant voltage can include a voltage of about 10 V, about 10 V to about 15 V, about 15 V to about 20 V, about 20 V to about 25 V, about 25 V to about 30 V, about 30 V to about 35 V, about 35 V to about 40 V, about 40 V to about 45 V, about 45 V to about 50 V, about 50 V to about 55 V, about 55 V to about 60 V, about 60 V to about 65 V, 65 V to about 70 V, 70 V to about 75 V, about 75 V to about 80 V, about 80 V to about 85 V, about 85 V to about 90 V, about 90 V to about 95 V, about 95 V to about 100 V, about 100 V to about 105 V, about 105 V to about 110 V, about 110 V to about 115 V, about 115 V to about 120 V, about 125 V to about 130 V, about 130 V to about 135 V, about 135 V to about 140 V, about 135 V to about 140 V, about 140 V to about 145 V, about 145 V to about 150 V, about 150 V to about 155 V, about 155 V to about 160 V, about 160 V to about 165 V, about 165 V to about 170 V and any increments therebetween.

In some embodiments, the electrical potential applied across the separating column is a varying electrical potential. For example, in some embodiments, an initial voltage or priming voltage can be applied across the separating column for a first increment of time including up to about 1 minute, from about 1 minute to about 2 minutes, from about 2 minutes to about 5 minutes, from about 5 minutes to about 8 minutes, from about 8 minutes to about 10 minutes, and any and all increments therebetween. The voltage can then be increased or decreased from the initial voltage to a secondary voltage.

In some embodiments, the initial voltage is less than the secondary voltage. In some embodiments, the initial voltage is greater than the secondary voltage. In some embodiments, the initial voltage includes a voltage of up to about 10 V, about 10 V to about 15 V, about 15 V to about 20 V, about 20 V to about 25 V, about 25 V to about 30 V, about 30 V to about 35 V, about 35 V to about 40 V, and any and all increments therebetween. In some embodiments, the secondary voltage includes a voltage that is greater than the initial or priming voltage.

In some embodiments, the secondary voltage includes a voltage of up to about 40 V, about 40 V to about 45 V, about 45 V to about 50 V, about 50 V to about 55 V, about 55 V to about 60 V, about 60 V to about 65 V, about 65 V to about 70 V, about 70 V to about 75 V, about 75 V to about 80 V, about 80 V to about 85 V, about 85 V to about 90 V, about 90 V to about 95 V, about 95 V to about 100 V about 100 V to about 105 V about 105 V to about 110 V, about 110 V to about 115 V, about 115 V to about 120 V, about 120 V to about 125 V, about 125 V to about 130 V, about 130 V to about 135 V, about 135 V to about 140 V, about 135 V to about 140 V, about 140 V to about 145 V, about 145 V to about 150 V, about 150 V to about 155 V, about 155 V to about 160 V, about 160 V to about 165 V, about 165 V to about 170 V and any and all increments therebetween.

In some embodiments, the electrical potential is pulsed on and off at a single level or at one or more levels for a set interval of time. For example, in some embodiments, a voltage is applied for up to about 30 seconds, from about 30 seconds to about 1 minute, from about 1 minute to about 2 minutes, from about 2 minutes to about 3 minutes, from about 3 minutes to about 4 minutes, from about 4 minutes to about 5 minutes, and so on, including all increments therebetween. In some embodiments, the pulsed or short-term electrical potential is a low voltage, for example about 25 V. In some embodiments, the pulsed or short-term electrical potential is an intermediate voltage, for example about 65 V. In some embodiments, the pulsed or short-term voltage is a high voltage, for example about 165 V. In some embodiments, the pulsed voltage is stepped up from a lower voltage to a higher voltage with subsequent pulses. In some embodiments, the pulsed or short-term electrical potential is applied at a first level and subsequent pulsed electrical potentials can include a greater or lesser level.

Embodiments of the methods include separating the biomolecule sample by molecular weight into at least n fractions, wherein n is any integer greater than zero, such as 1, 2, 3, 4, 5, 6, 7, 8, 9, 10, and the like. The sample can be separated into from about 2 to about, for example, 2000 fractions, from about 2 fractions, up to about 4 fractions, up to about 8 fractions, up to about 12 fractions, up to about 24 fractions, up to about 36 fractions, up to about 48 fractions, up to about 96 fractions, up to about 384 fractions, up to about 1536 fractions, and any and all increments therebetween.

The sample is separated by applying an electrical potential to the column and dispensing each of the n fractions from the bottom of the column into separate, distinct wells, wherein each well receives a fraction. In embodiments, the separating column is advanced to a different well and placed in fluid communication with the fractionation buffer in that well. In some embodiments, each well receives one fraction. In some embodiments, one or more wells receive more than one fraction.

In some embodiments, the n fractions can be determined by one or more means including (1) by observing the advancement of a molecular weight marker along the separating column, (2) by applying an electrical potential for a predetermined interval of time, by (3) observing the advancement of a molecular weight marker in a reference column, or a combination thereof.

In some embodiments, the advancement of a molecular weight marker along the separating column is observed visually by a user wherein the electrical potential is applied until a specific band or known standard in the molecular weight marker is observed to advance to a position in the separating column; in some embodiments, this is accomplished in an automated fashion, for example, via an automated imaging system or using one or more detectors or sensors—such as optical detectors, sensors, and the like—to provide feedback to a central control unit or automation unit. The position may include the bottom of the separating column. The position may include a position beyond the end of the separating column including advanced and expunged from the bottom of the separating column. The position may include an intermediate position along the separating column. In some embodiments, the advancement of a molecular weight marker along the separating column is detected by a detector. The detector can detect an optical signal including a colorimetric signal, a fluorescence signal, a bioluminescence signal or the like that corresponds to a position or advancement of sample separated along the separating column.

In some embodiments, the advancement of the sample along the separating column and the separation of the sample into one or more fractions is determined by applying an electrical potential to the separating column for a predetermined or prescheduled interval of time. For example, in some embodiments, a voltage is applied across the separating column for a first interval of time and the first fraction corresponds to the amount of sample dispensed into the first well during the first interval of time.

Similarly, in some embodiments, the column is advanced to a second well and an electrical potential is applied across the separating column for a second interval of time dispensing a second fraction corresponding to the amount of sample dispensed into the second well during the second interval. In some embodiments, the first interval and second interval are the sample amount of time. In some embodiments, the first interval and second interval are different amounts of time. In some embodiments, the steps of advancing the column to a new well and applying an electrical potential for an interval of time thereby dispensing a fraction are repeated until the sample is fractionated and dispensed into wells.

In some embodiments the first, second or subsequent intervals of time can include an amount of time of up to 30 seconds, from about 30 seconds to about 60 seconds, from about 1 minute to about 2 minutes, from about 2 minutes to about 3 minutes, from about 3 minutes to about 4 minutes, from about 4 minutes to about 5 minutes, from about 5 minutes to about 6 minutes, from about 6 minutes to about 7 minutes from about 7 minutes to about 8 minute, from about 8 minutes to about 9 minutes, from about 9 minutes to about 10 minutes and any and all increments therebetween.

In some embodiments, a second column is loaded in parallel to the separating column wherein the second column is a reference column. In some embodiments, the reference column is identical to the separating column, contains the same separating medium, is loaded with one or more molecular weight markers, and is loaded with the same volume of cathode buffer or running buffer including but is not loaded with a biomolecule sample. In such embodiments, the molecular weight marker in the reference column is used as an indicator of the progression of the biomolecule sample through the separating medium of the separating column.

As such, the reference column is used as a guide to identify fractions. In some embodiments, the reference column is used to determine the size or volume of the fractions. In some embodiments, the reference column is used to determine when to advance to a new well. In some embodiments, the new well is an adjacent well. In some embodiments, the new well is in a different location on the plate.

Embodiments of the methods include forming x distinct fractions by contacting each of the n fractions with a distinct microparticle. In some embodiments, the microparticle can comprise one or more of a magnetic material, a fluorescent label, a colorimetric label, a bioluminescent label, a protein, a polymer—which polymer can be a plastic polymer, a dye, and/or one or more combinations thereof. In some embodiments, the microparticle includes a bead such as a magnetic bead. In some embodiments, the microparticle has a modified surface. For example, in some embodiments, the microparticle is biotinylated, carboxylated, or includes one or more other surface treatments including ANTEOBIND™; other coupling methods including covalent chemistries can be used. In some embodiments, the microparticle includes one or more shapes including a sphere, cylinder, disc or other suitably shaped particle, as understood in the art. In some embodiments, a microparticle may be labeled with a distinct bar code, one or more distinct dyes, one or more distinct labels including fluorescent labels, colorimetric labels, chemiluminescent labels, bioluminescent labels or other similar labels for distinguishing subpopulations of microparticles. A microparticle can also comprise a distinct protein; microparticles can also be distinct based on size; for example, microparticles can be of two or more sizes. Microparticles can also be distinct based on density; for example, microparticles can be of two or more different densities. In some embodiments, the n fractions are formed into x distinct fractions wherein x is less than n. That is, in some embodiments, more than one of the n fractions is labeled with the same distinct microparticle thereby forming a subset of similarly labeled fractions. In some embodiments, the n fractions are formed into x distinct fractions wherein x is equal to n.

In some embodiments, the labeled microparticles are preloaded into the one or more separation wells. In some embodiments, the labeled microparticles are added to the wells after the n fractions are dispensed into the fraction wells.

In some embodiments, the n fractions are contacted with a dilution buffer. In some embodiments, the n fractions are contacted with a dilution buffer before the microparticles are added to the wells. In some embodiments, the n fractions are contacted with a dilution buffer after the microparticles are added to the wells; the dilution buffer can also be added as the fractions are contacted with the microparticles. In some embodiments, the dilution buffer can include one or more of buffering agents, salts, non-ionic detergents, and the like. For example, in some embodiments, the dilution buffer includes one or more of Tris, sodium chloride, a detergent, and combinations thereof. A detergent can include a nonionic surfactant, an ionic surfactant, a zwitterionic surfactant—such as CHAPS, for example—digitonins, and combinations thereof. Example nonionic surfactants include, without limitation, Tween surfactants, such as Tween20; Triton surfactants, such as Triton X-100; and the like.

Embodiments of the methods include combining the x distinct fractions into a fraction pool. The pooled fraction can be pooled into a pooling container. In some embodiments, the fraction pool is distributed into a second multi-well plate where equal amounts of the fraction pooled are deposited into wells of the multi-well plate. The equal amounts of the fraction pool can include up to about 1 μL, from about 1 μL to about 2 μL, from about 2 μL to about 4 μL, from about 4 μL to about 6 μL, from about 6 μL to about 8 μL, from about 8 μL to about 10 μL, from about 10 μL to about 12 μL, from about 12 μL to about 14 μL, from about 14 μL to about 16 μL, from about 16 μL to about 18 μL, from about 18 μL to about 20 μL and any and all increments therebetween.

Embodiments of the methods also include contacting a first amount of the fraction pool with a first primary antibody. In some embodiments, the first primary antibody is a labeled primary antibody, though this is not a requirement. The primary antibody can be labeled with one or more of a fluorescence label, a bioluminescence label, a colorimetric label, or one or more combinations thereof. In some embodiments, the first primary antibody includes an unlabeled primary antibody.

Embodiments of the methods include determining a presence or absence of a biomolecule in the first amount of the fraction pool by detecting the presence of absence of the first primary antibody bound to the biomolecule in the first amount of the fraction pool. The first primary antibody may be detected using any suitable means including for example an optical detector for detecting the amount of fluorescent, bioluminescent or colorimetric label present. The first primary antibody may be detected using other suitable means for measuring the labeled microparticle and the labeled primary antibody in order to determine the molecule weight of the detected biomolecule. Embodiments of the methods include further contacting the fraction pool with one or more additional primary antibodies for detecting more than one biomolecules in the sample.

Embodiments of the methods can include contacting the first amount of the fraction pool with a first secondary antibody that can specifically bind to the first primary antibody. In some embodiments, the first primary antibody in an unlabeled primary antibody. In some embodiments, the first secondary antibody is a labeled secondary antibody. The secondary antibody can be labeled with one or more of a fluorescence label, a bioluminescence label, a colorimetric label, or one or more combinations thereof. In some embodiments, the fraction pool is contacted with more than one secondary antibody for detecting more than one primary antibody contacted with the biomolecules in the fraction pool.

Embodiments of the methods can also include determining a presence or absence of a biomolecule in the fraction pool, the determining being based on the presence or absence of first secondary antibody bound to first primary antibody bound to the biomolecule in the first amount of the fraction pool.

### Examples

The following examples are illustrative only and do not limit the scope of the disclosed technology.

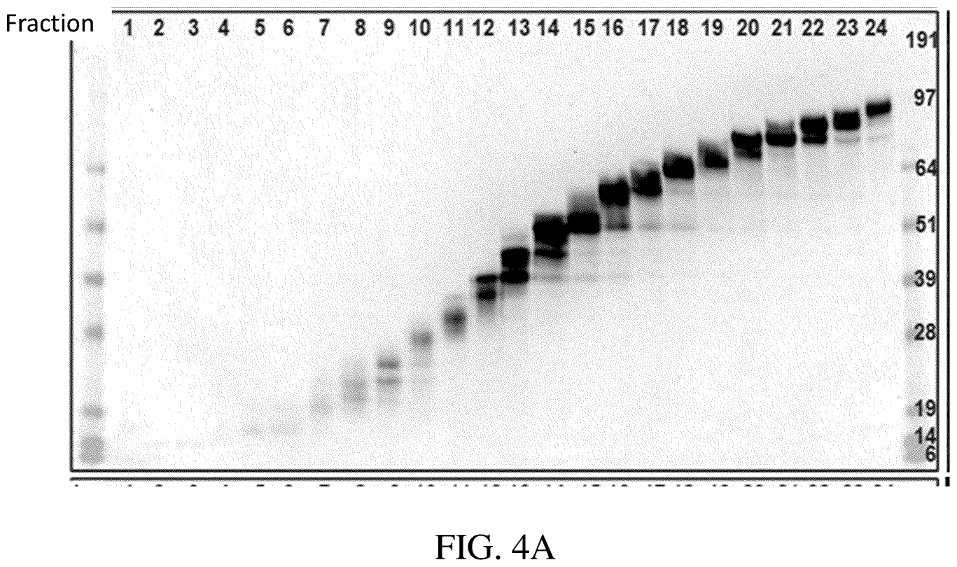

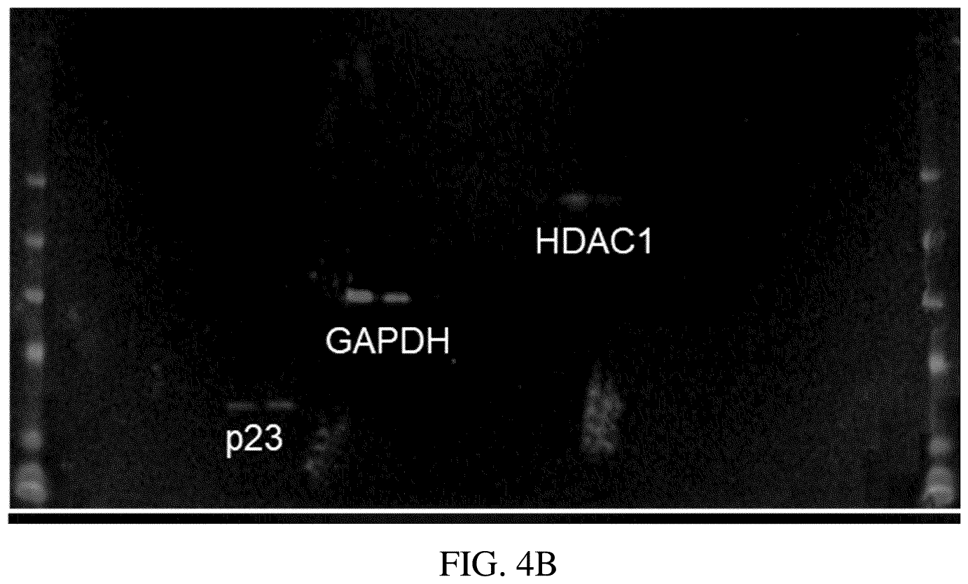

FIGS. 4A and 4B depict a validating Western blot analysis of protein fractions after sample fractionation using the separation column of the present disclosure. FIG. 4A depicts a membrane treated with a total protein stain where the membrane contains fractions of a protein sample separated using a separation column of the disclosed technology. FIG. 4B depicts a Western blot showing detection of p23, GAPDH, and HDAC1 at their respective molecular weights, on a membrane containing fractions of a protein sample that was separated using a separation column and fractionation methods of the disclosed technology. The sample was fractionated according to the methods of the present disclosure and the fractions were loaded into a gel and electrophoretically run along the gel The fractionated proteins were then transferred to a membrane for detecting the fractions using standard Western blotting techniques. The blotted membranes—both total protein (FIG. 4A) and specific proteins (FIG. 4B)—demonstrate the fractionated samples each contain proteins at distinct and incremental molecular weights.

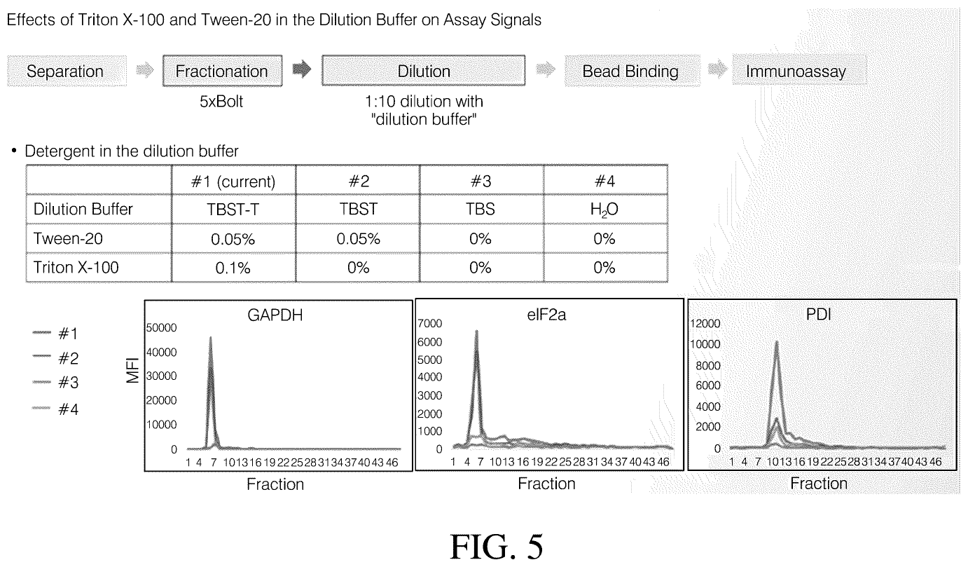

FIG. 5 illustrates the effect on assay signals from varying the surfactant in an example dilution buffer when using the disclosed technology. FIG. 5 illustrates the effect of differently-composed dilution buffers, more specifically the effect of varying the Triton X-100 and Tween-20 content in the buffer. As shown, the use of buffer #1 resulted in the best sequestration by weight fraction for each of three proteins—GAPDH, elF2a, and PDI—as shown by fraction vs. MFI signal; as seen, buffers #2, #3, and #4 did not exhibit the degree of protein sequestration as buffer #1. Without being bound to any particular theory, TBS containing 0.05% Tween-20 and 0.1% Triton X-100 exhibited the best performance.

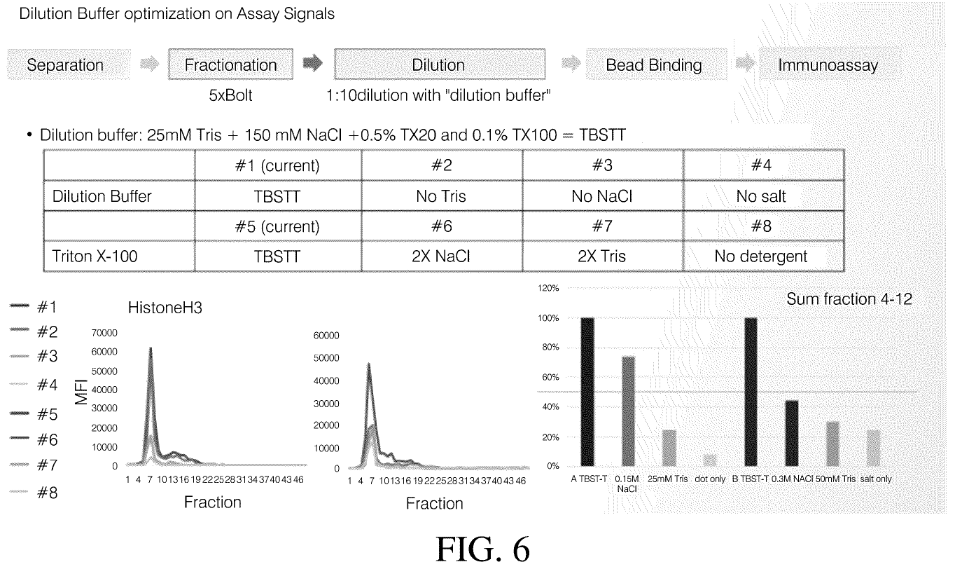

FIG. 6 illustrates the effect on assay signals from varying the composition of an example dilution buffer when using the disclosed technology. The various buffer compositions of FIG. 6 are provided below:

TABLE 1
Exemplary Dilution Buffers with Triton X-100

| Dilution Buffer | Amount | Amount of | Amount of | Amount of |
|---|---|---|---|---|
| Sample No. | of Tris | NaCl | Tween 20 | Triton X-100 |
| 1 | 25 mM | 150 mM | 0.5% | 0.1% |
| 2 | — | 150 mM | 0.5% | 0.1% |
| 3 | 25 mM | — | 0.5% | 0.1% |
| 4 | 25 mM | — | 0.5% | 0.1% |
| 5 | 25 mM | 150 mM | 0.5% | 0.1% |
| 6 | 25 mM | 300 mM | 0.5% | 0.1% |
| 7 | 50 mM | 150 mM | 0.5% | — |
| 8 | — | 150 mM | — | — |

As shown, the use of buffer #1 resulted in the best sequestration by weight fraction for Histone H3 as shown by fraction vs. MFI signal; as seen, buffers #2, #3, #4, #6, #7, and #8 did not exhibit the degree of protein sequestration as buffers #1 and #5. As shown by the bar charts at the right of FIG. 6, buffers #1 and #5 (i.e., TBSTT) exhibited the best performance as shown by their superior sequestration of the protein in weight fractions 4-12. Without being bound to any particular theory, buffer containing some salt and some detergent (preferably more than 50 mM salt) exhibited the best performance.

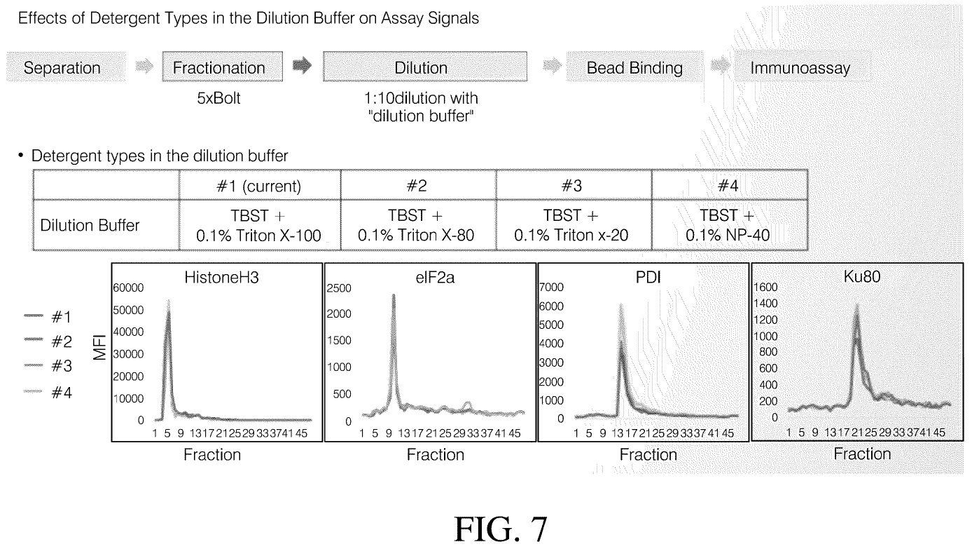

FIG. 7 illustrates the effect on assay signals from varying the detergent in an example dilution buffer when using the disclosed technology. The various buffer compositions of FIG. 7 are provided below:

TABLE 2
Dilution Buffers with varying detergents

| Dilution | Amount | Amount | Amount | Amount of |
|---|---|---|---|---|
| Buffer Sample | of | of | of | Additional |
| No. | Tris | NaCl | Tween 20 | Detergent |
| 1 | 25 mM | 150 mM | 0.5% | 0.1% Triton |
| | | | | X-100 |
| 2 | 25 mM | 150 mM | 0.5% | 0.1% Triton |
| | | | | X-80 |
| 3 | 25 mM | 150 mM | 0.5% | 0.1% Triton |
| | | | | X-20 |
| 4 | 25 mM | 150 mM | 0.5% | 0.1% NP-40 |

As shown, the use of buffer #1 resulted in the best sequestration by weight fraction for each of four proteins (Histone H3, elF2a, PDI, and Ku80) as shown by fraction vs. MFI signal; as seen, buffers #2, #3, and #4 did not exhibit the degree of protein sequestration as buffer #1. Without being bound to any particular theory, Triton X-100 in TBSTT can be replaced by other types of detergent while still achieving relatively the same protein retention in a narrow band of fractions. A detergent can include, for example, any one or more of a nonionic surfactant, an ionic surfactant; a zwitterionic surfactant, such as CHAPS; and digitonins. Example nonionic surfactants include, for example, Tween surfactants, such as Tween20; Triton surfactants, such as Triton X-100; and the like.

It should be understood that the example buffer formulations provided herein are illustrative only and are not exclusive, as other buffer formulations are within the scope of the disclosed technology.

It should also be understood that although some focus was placed on separation and assay of cell lysates, for example, 1-10 μg of treated and untreated full cell lysates, the disclosed method can also be applied to more or less protein. Further, the separation conditions and assays performance can be modified if used in combination with alternative sample types. Such alternative sample types include those that have been simplified through some time of enrichment, such as organelle isolation, surface protein isolation, phosphoenrichment, or immunoprecipitation/co-immunioprecipitation.

What is claimed:

1. A method for fractionating a sample of biomolecules, comprising:
a. introducing the sample into a separating column;
b. separating biomolecules in the sample by molecular weight into at least n fractions along the separating column; and
c. placing the separating column into successive engagement with first through nth wells and advancing into each of the first through nth wells the corresponding first through nth fraction.

2. The method of claim 1, wherein at least two of the first through nth wells are comprised in a multi-well container.

3. The method of any one of claims 1-2, wherein the separating column is removed from engagement from a well based at least in part on any one or more of (a) observation of the separating column, (b) a predetermined time interval, and (c) observation of a reference column containing a marker.

4. The method of claim 3, wherein observation of the separating column comprises observing movement of a molecular weight marker, a size standard, a dye, a fluorescent marker, a colorimetric marker, or any combination thereof.

5. The method of claim 3, wherein observation of the reference column comprises observing movement of a molecular weight marker, a size standard, a dye, a fluorescent marker, a colorimetric marker, or any combination thereof.

6. The method of any one of claims 1-5, wherein placing the separating column into engagement with a well comprises moving at least one of the separating column or the well.

7. The method of any one of claims 1-6, wherein separating biomolecules in the sample by molecular weight comprises applying an electrical potential across the sample.

8. The method of claim 7, wherein the electrical potential is constant.

9. The method of claim 7, wherein the electrical potential is variable.

10. The method of any one of claims 1-9, wherein a biomolecule of the sample comprises any of a protein, a nucleic acid, a carbohydrate, or a lipid.

11. The method of any one of claims 1-10, wherein the separating column comprises a channel at least partially filled with a solid or liquid separation matrix, such as Polymerized acylamide (SDS-PAGE), Linear acrylamide, PDMA, or dextran.

12. The method of claim 11, wherein the separating column (a) is cylindrical, square, or rectangular in conformation, (b) has an internal diameter in the range of from about 0.1 mm to about 1 mm, (c) has a variable internal cross-section along the length of the separating column, or (d) any two or more of (a), (b), and (c).

13. The method of any one of claims 1-12, wherein the separating column comprises a separating medium.

14. The method of claim 13, wherein the separating medium comprises a separating gel or liquid matrix.

15. The method of claim 14, wherein the separating gel comprises a Tris-glycine gel, a Bis-Tris gel, a Tris-acetate gel, a Tricine gel, a polyacrylamide gel, an acrylamide gel, an agarose gel, or any combination thereof.

16. The method of claim 13, wherein the separating medium further comprises a stacking gel.

17. The method of claim 16, wherein the stacking gel comprises a polyacrylamide gel.

18. The method of any one of claims 1-17, wherein n is in a range of from 2 to about 1536.

19. The method of any one of claims 1-18, further comprising contacting one or more of the n fractions with a distinct microparticle each to form one or more distinct fractions, wherein each distinct fraction is different from other distinct fractions.

20. The method of claim 19, wherein x distinct fractions are formed by contacting x fractions with x distinct microparticles, x being less than or equal to n.

21. The method of any one of claims 19-20, wherein the microparticle comprises a magnetic material, a fluorescent material, a bar code, a dye, a label, a protein, a polymer, or a combination thereof.

22. The method of any one of claims 19-21, wherein the distinct microparticle comprises any one or more of a distinct barcode, a distinct fluorescent label, a distinct colorimetric label, a distinct bioluminescent label, a distinct dye label, a protein, a distinct density, or a distinct size.

23. The method of claim 19, wherein a pre-labeled or labeled fraction is contacted with one or more buffers.

24. The method of claim 23, wherein the one or more buffers comprise a dilution buffer.

25. The method of claim 24, wherein the dilution buffer comprises any one or more of Tris, sodium chloride, and a detergent.

26. The method of any one of claims 20-25, further comprising combining the x distinct fractions to form a fraction pool.

27. The method of claim 26, wherein the biomolecule comprises a protein, and further comprising contacting a first amount of the fraction pool with a first primary antibody.

28. The method of claim 27, wherein the first primary antibody is a labeled primary antibody.

29. The method of any one of claims 26-28, further comprising determining a presence or absence of a biomolecule in the first amount of the fraction pool, the determining being based on the presence or absence of first primary antibody bound to the biomolecule in the first amount of the fraction pool.

30. The method of claim 29, further comprising contacting the first amount of the fraction pool with a first secondary antibody that can bind complementarily to the first primary antibody, wherein the first secondary antibody is a labeled secondary antibody.

31. The method of claim 30, further comprising determining a presence or absence of a biomolecule in the fraction pool, the determining being based on the presence or absence of first secondary antibody bound to first primary antibody bound to the biomolecule in the first amount of the fraction pool.

32. The method of claim 26, further comprising contacting a second amount of the fraction pool with a second primary antibody.

33. The method of claim 32, wherein the second primary antibody is a labeled primary antibody.

34. The method of any one of claims 32-33, further comprising determining a presence or absence of a biomolecule in the second amount of the fraction pool, the determining being based on the presence or absence of second primary antibody bound to the biomolecule in the second amount of the fraction pool.

35. The method of claim 34, further comprising contacting the second amount of the fraction pool with a second secondary antibody that can bind complementarily to the second primary antibody, wherein the second secondary antibody is a labeled secondary antibody.

36. The method of claim 35, further comprising determining a presence or absence of a biomolecule in the fraction pool, the determining being based on the presence or absence of second secondary antibody bound to second primary antibody bound to the biomolecule in the second amount of the fraction pool.

37. The method of claim 26, wherein the biomolecule comprises a nucleic acid, and wherein the method further comprises contacting a first amount of the fraction pool with a first complementary nucleic acid probe.

38. The method of claim 37, wherein the first complementary nucleic acid probe comprises a detectable label.

39. The method of any one of claims 37-38, further comprising determining a presence or absence of a biomolecule in the first amount of the fraction pool, the determining being based on the presence or absence of the first complementary nucleic acid probe bound to the biomolecule in the first amount of the fraction pool.

40. The method of claim 26, wherein the biomolecule comprises a nucleic acid, and wherein the method further comprises performing a bDNA assay to determine the presence or absence of the biomolecule in the first amount of the fraction pool.
